(19) United States
(12) Patent Application Publication
(10) Pub. No.: US 2015/0006200 A1
(43) Pub. Date: Jan. 1, 2015

(54) SYSTEM AND METHOD FOR PROVIDING AUTOMATED HOME-BASED HEALTH SERVICES

(71) Applicants: Robert Joseph Chaput, Garson (CA); Maneesh Walia, Sudbury (CA); Alain Adrien Leclair, Sudbury (CA)

(72) Inventors: Robert Joseph Chaput, Garson (CA); Maneesh Walia, Sudbury (CA); Alain Adrien Leclair, Sudbury (CA)

(21) Appl. No.: 13/931,154

(22) Filed: Jun. 28, 2013

(51) Int. Cl. G06Q 50/22; G06Q 10/06

(52) U.S. Cl. CPC G06Q 50/22; G06Q 10/063118
USPC 705/3

(57) ABSTRACT

A method and system are provided for coordinating caregiving services for clients and care workers. The method and system involve storing at least one client record containing client particulars and at least one care worker record containing care worker particulars; operating at least one processor for, upon receiving a client attention request, associating the client attention request with a client record, selecting a ranked care worker record, creating a homecare visit record, providing notice of existence of the homecare visit record to the selected care worker, and upon receiving a care worker accept decision signal, associating the client attention request with the selected care worker record, and providing notice of transfer of the homecare visit record to the selected care worker.

### FIELD

The described embodiments relate to methods and systems for providing caregiving services and in particular, for coordinating caregiving services for clients and care workers.

### INTRODUCTION

A large segment of the population depends on caregiving services and products to sustain safe, healthy, and independent lives while continuing to live in their own home. Caregiving companies often provide a variety of services including nursing, home support, and personal care services.

Each client is unique. Each client may have particular preferences, preferences relating to language and culture. Each client may have unique characteristics, such as characteristics relating to health or abilities. Similarly, each individual care worker is unique. Each care worker will differ from their care worker colleagues. For example, each care worker may have different specialties, caregiving service abilities, or language abilities.

Historically, systems for managing and coordinating caregiving services may focus on matching clients having particular physical features, characteristics, or symptoms with treatment options based on aggregate data from clients with similar features, characteristics, or symptoms. Similarly, systems for managing and coordinating caregiving services may focus on assigning care workers to a caregiving role based on the qualification requirements of that role.

In some instances, generalizing patient treatment options may be sufficient. Similarly, in some instances, assigning care workers to a generalized caregiving role may be sufficient. However, with an increasingly diverse client population, it may not be sufficient to service client requirements with generalized treatment options and with generalized caregiving roles.

### SUMMARY

The various embodiments described herein generally relate to methods (and associated systems configured to implement the methods) for coordinating caregiving services for clients and care workers. The method comprising storing at least one client record containing client particulars and at least one care worker record containing care worker particulars in the database; upon receiving a client attention request, associating the client attention request with a client record, wherein the client attention request includes a requested caregiving procedure, selecting a ranked care worker record based on the client attention request, the client record, and the care worker records, creating a homecare visit record based on the client attention request, the client record, and the selected care worker record and storing the homecare visit record in the database, wherein the homecare visit record further comprises a record of caution signals and alarm response signals, providing notice of existence of the homecare visit record to the selected care worker associated with the care worker record; upon receiving a care worker accept decision signal, associating the client attention request with the selected care worker record, and providing notice of transfer of the homecare visit record to the care worker associated with the selected care worker record.

In accordance with an embodiment of the invention, there is provided a system for coordinating caregiving services for clients and care workers. The system comprising a database for storing at least one client record containing client particulars and at least one care worker record containing care worker particulars; a processor and selection module coupled to the database, said processor operable to: upon receiving a client attention request, associate the client attention request with a client record, wherein the client attention request includes a requested caregiving procedure, select a ranked care worker record based on the client attention request, the client record, and the care worker records, create a homecare visit record based on the client attention request, the client record, and the selected care worker record and store the homecare visit record in the database, wherein the homecare visit record further comprises a record of caution signals and alarm response signals, provide notice of existence of the homecare visit record to the selected care worker associated with the care worker record; upon receiving a care worker accept decision signal: associate the client attention request with the selected care worker record, and provide notice of transfer of the homecare visit record to the care worker associated with the selected care worker record.

In accordance with an embodiment of the invention, there is provided a non-transitory computer-readable medium comprising instructions executable on at least one processor for implementing a method for coordinating caregiving services for clients and care workers using a caregiving services computing system comprising a database and processor. The method comprising storing at least one client record containing client particulars and at least one care worker record containing care worker particulars in the database; upon receiving a client attention request, associating the client attention request with a client record, wherein the client attention request includes a requested caregiving procedure, selecting a ranked care worker record based on the client attention request, the client record, and the care worker records, creating a homecare visit record based on the client attention request, the client record, and the selected care worker record and storing the homecare visit record in the database, wherein the homecare visit record further comprises a record of caution signals and alarm response signals, providing notice of existence of the homecare visit record to the selected care worker associated with the care worker record; upon receiving a care worker accept decision signal, associating the client attention request with the selected care worker record, and providing notice of transfer of the homecare visit record to the care worker associated with the selected care worker record.

Further aspects and advantages of the embodiments described herein will appear from the following description taken together with the accompanying drawings.

### DRAWINGS

For a better understanding of embodiments of the systems and methods described herein, and to show more clearly how they may be carried into effect, reference will be made, by way of example, to the accompanying drawings in which:

FIG. 1 is a block diagram of a system for coordinating caregiving services for clients and care workers in accordance with at least one embodiment;

FIG. 2 is a schematic diagram of a database of a system for coordinating caregiving services of FIG. 1 in accordance with at least one embodiment;

FIG. 3 is a schematic diagram of a collection of modules of a system for coordinating caregiving services of FIG. 1 in accordance with at least one embodiment;

FIG. 4 is a flowchart of an example embodiment of a method for coordinating caregiving services involving a client, a selected care worker, and the system of FIG. 1 in accordance with at least one embodiment;

FIG. 5 is a flowchart of an example embodiment of a method for identifying and flagging complexities that could arise when providing a requested caregiving procedure to a client with particular characteristics or medical history;

FIG. 6 is a flowchart of an example embodiment of a method for matching a client record with at least one care worker record in accordance with at least one embodiment;

FIG. 7 is a block diagram of an example embodiment for performing a correlation search and compare of a client record and at least one care worker record in accordance with at least one embodiment;

FIG. 8 is a flowchart of an example embodiment for a method of setting up allocated time parameters and tracking the amount of time a care worker utilizes for a caregiving visit in accordance with at least one embodiment;

FIG. 9 is a screenshot of a user interface where a care worker may review contents of a homecare visit record in accordance with an example embodiment;

FIG. 10 is a screenshot of a user interface for accessing client data of a client record by a user on a user device in accordance with an example embodiment;

FIG. 11 is a screenshot of a user interface for accessing client data of a client record by a user on a user device in accordance with another example embodiment; and

FIG. 12 is a screenshot of a user interface for accessing client data of a client record by a user on a user device in accordance with another example embodiment.

### DESCRIPTION OF VARIOUS EMBODIMENTS

The various embodiments described herein generally relate to methods (and associated systems configured to implement the methods) for coordinating caregiving services for clients and care workers.

It will be appreciated that numerous specific details are set forth in order to provide a thorough understanding of the example embodiments described herein. However, it will be understood by those of ordinary skill in the art that the embodiments described herein may be practiced without these specific details. In other instances, well-known methods, procedures and components have not been described in detail so as not to obscure the embodiments described herein. Furthermore, this description and the drawings are not to be considered as limiting the scope of the embodiments described herein in any way, but rather as merely describing the implementation of the various embodiments described herein. Where considered appropriate, for simplicity and clarity of illustration, reference numerals may be repeated among the figures to indicate corresponding or analogous elements or steps.

The embodiments of the systems and methods described herein may be implemented in hardware or software, or a combination of both. These embodiments may be implemented in computer programs executing on programmable computers, each computer including at least one processor, a data storage system (including volatile memory or non-volatile memory or other data storage elements or a combination thereof), and at least one communication interface. For example, a suitable programmable computer may be a server, network appliance, embedded device, computer expansion module, personal computer, laptop, or any other computing device capable of being configured to carry out the methods described herein.

In some embodiments, the communication interface may be a network communication interface. In embodiments in which elements are combined, the communication interface may be a software communication interface, such as those for inter-process communication (IPC). In still other embodiments, there may be a combination of communication interfaces implemented as hardware, software, and combination thereof.

Program code may be applied to input data to perform the functions described herein and to generate output information. The output information is applied to one or more output devices, in known fashion.

Each program may be implemented in a high level procedural or object oriented programming and/or scripting language, or both, to communicate with a computer system. However, the programs may be implemented in assembly or machine language, if desired. In any case, the language may be a compiled or interpreted language. Each such computer program may be stored on a storage media or a device (e.g. ROM, magnetic disk, optical disc) readable by a general or special purpose programmable computer, for configuring and operating the computer when the storage media or device is read by the computer to perform the procedures described herein. Embodiments of the system may also be considered to be implemented as a non-transitory computer-readable storage medium, configured with a computer program, where the storage medium so configured causes a computer to operate in a specific and predefined manner to perform the functions described herein.

Furthermore, the system, processes and methods of the described embodiments are capable of being distributed in a computer program product comprising a computer readable medium that bears computer usable instructions for one or more processors. The medium may be provided in various forms, including one or more diskettes, compact disks, tapes, chips, wireline transmissions, satellite transmissions, internet transmission or downloadings, magnetic and electronic storage media, digital and analog signals, and the like. The computer useable instructions may also be in various forms, including compiled and non-compiled code.

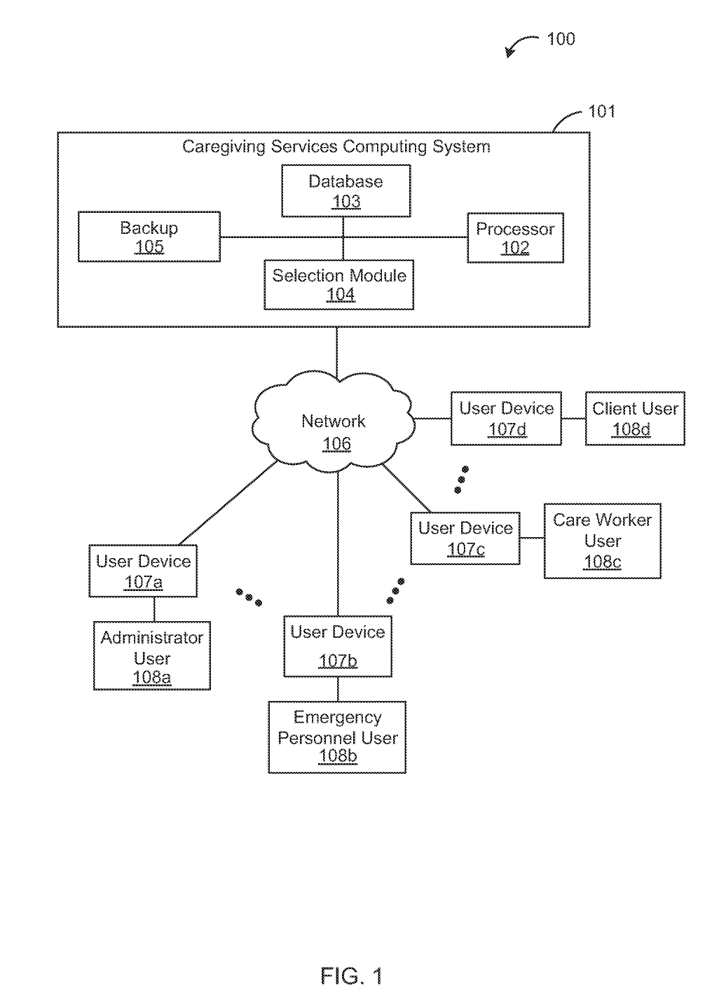

Reference is first made to FIG. 1 which shows a system for coordinating caregiving services 100 made in accordance with a preferred embodiment. The system 100 comprises caregiving services computing system 101, a network 106, user devices 107, and users 108. Caregiving services computing system 101 comprises a processor 102, a database 103, a selection module 104, and backup 105.

Caregiving services computing system 101 may be implemented using, for example, a general-purpose computer capable of responding to and executing instructions in a defined manner, a personal computer, a special purpose computer, a workstation, a server, a device, a component, or other equipment or some combination thereof capable of responding to and executing instructions. While caregiving services computing system 101 is described as comprising processor 102, database 103, selection module 104, and backup 105, it will be understood by those skilled in the art that the caregiving services computing system 101 may comprise any number of processors, databases, selection modules, and backups to implement the system.

User devices 107 are connected to caregiving services computing system 101 via a network 106. Network 106 may be implemented by various well known communication networks, including a local area network (LAN), a wide area network (WAN), the Internet, analog or digital wired and wireless telephone networks (e.g. a Public Switched Telephone Network (PSTN), an Integrated Services Digital Network (ISDN), or a Digital Subscriber Line (xDLS), or any other wired or wireless network. The network 106 may include multiple networks or sub-networks, each of which may include, for example, a wired or wireless communication channel.

In an embodiment, user device 107a may be operated by, for example, an administrator user 108a who has access to and control of all data in database 103. An administrator user 108a may include registered nurses, registered practical nurses, personal support workers, caregiving services administrators, or department managers.

User device 107b may be operated by, for example, an emergency personnel user 108b. In an embodiment, emergency personnel user 108b may access data in database 103 when dispatched to a client's location for emergency response.

User device 107c may be operated by, for example, a care worker user 108c who has access rights to view and modify data in database 103. A care worker user 108c may include caregiving professionals, registered nurses, registered practical nurses, personal support workers, or pharmacists.

User device 107d may be operated by, for example, a client user 108d with limited access to data in database 103. Client user 108d may access the database to verify the accuracy of data. Client user 108d may also have limited access rights to input data into database 103. A client user 108d may input data by completing questionnaires and feedback forms.

It will be appreciated that the system for coordinating caregiving services 100 is not limited to user devices 107a, 107b, 107c, and 107d and users 108a, 108b, 108c, and 108d. The system for coordinating caregiving services may comprise an unlimited number of user devices 107 operated by any number of various users 108. Further, it is contemplated that users 108 may access network 106 from a multiple and a variety of different devices 107 and need not use the same device 107 each time user 108 accesses the system for coordinating caregiving services 101 via network 106.

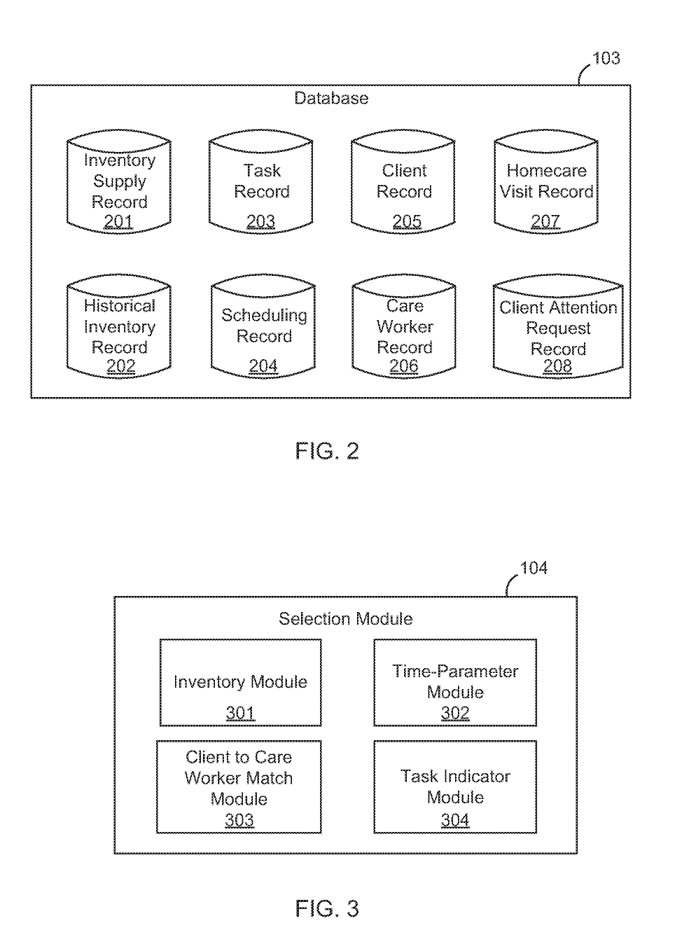

Reference is now made to FIG. 2, in which an exemplary embodiment of database 103 of the caregiving services computing system 101 of FIG. 1 is illustrated. In an embodiment, database 103 may contain inventory supply record 201, historical inventory record 202, task record 203, scheduling record 204, client record 205, care worker record 206, homecare visit record 207, and client attention request record 208. It will be appreciated that the various elements in database 103 are not limited to single records. The various records may comprise an unlimited number of records storing an unlimited type and amount of data.

Inventory supply record 201 may contain data fields with information tracking consumable medical supplies including bandages, diagnostic equipment, instruments, and disposable garments.

Historical Inventory record 202 may contain inventory usage data. The data may be summarized in aggregate and may be used to predict future inventory supply requirements and to provide statistical data necessary for cost control analysis.

In an embodiment, task record 203 may contain licensed versions of approved caregiving procedures and definitions. In another exemplary embodiment, the task record 203 may contain indication data, where indication data includes information that may identify and flag incompatibilities or complexities between a requested caregiving procedure and client particulars.

Scheduling record 204 may contain appointment details including location of a caregiving appointment, time of caregiving appointment, time allocated for the caregiving appointment, client contact information on the day of the caregiving appointment, listing of caregiving services planned for the caregiving appointment, listing of care workers scheduled to attend, and special instructions for the caregiving appointment.

Client record 205 may contain individualized client data including client personal information, client health information, and client care charts.

Care worker record 206 may contain individualized care worker data including information on the care worker's past professional experiences, expertise, specialized training or accreditation, language abilities, travel availability, gender, and ability to understand and accommodate cultural and religious preferences.

Homecare visit record 207 may be based on a client attention request record 208, a client record 205, and care worker records 206. In an embodiment, homecare visit records organize all pertinent information relevant to a requested caregiving procedure. In addition, homecare visit records may store flags or signals that may be raised before, during, or after a caregiving service appointment.

Reference is now made to FIG. 3, in which an exemplary embodiment of selection module 104 of the caregiving services computing system 101 of FIG. 1 is illustrated. The selection module 104 may contain inventory module 301, time-parameter module 302, client to care worker match module 303, and task indicator module 304.

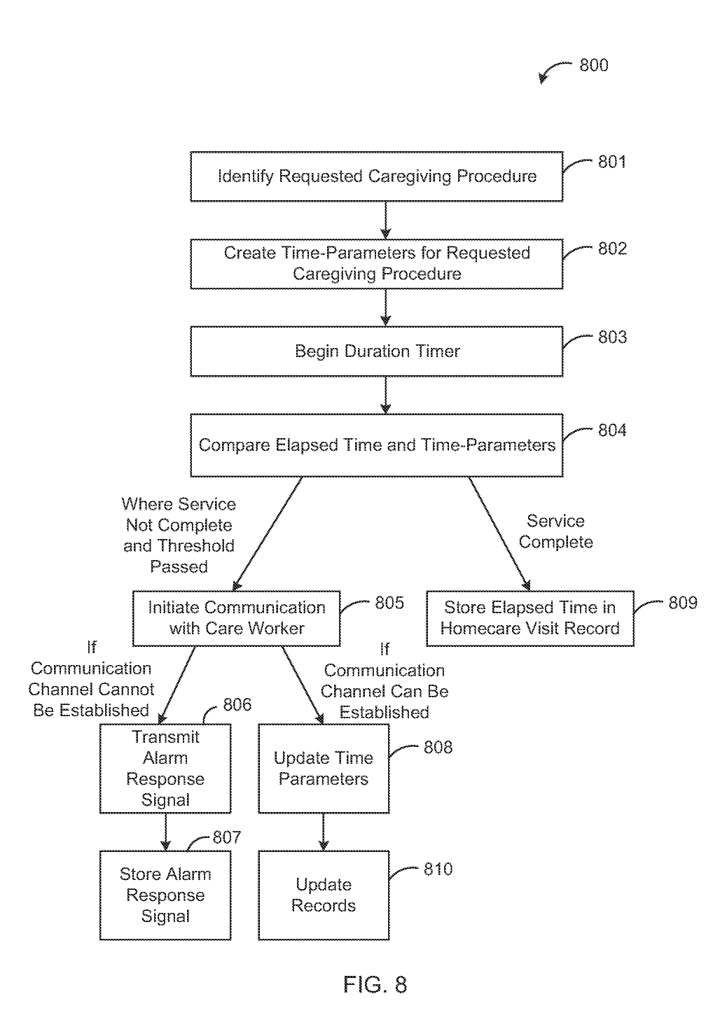

Inventory module 301 may be used to organize inventory data, analyze inventory data, and predict future inventory data usage. Time-parameter module 302 may set up time parameters for each caregiving service appointment, may keep track of each caregiving service appointment, and may send alarm signals in response to caregiving service appointments exceeding time allocated by time parameters. Time-parameter module 302 will be discussed in further detail having regard to FIG. 8.

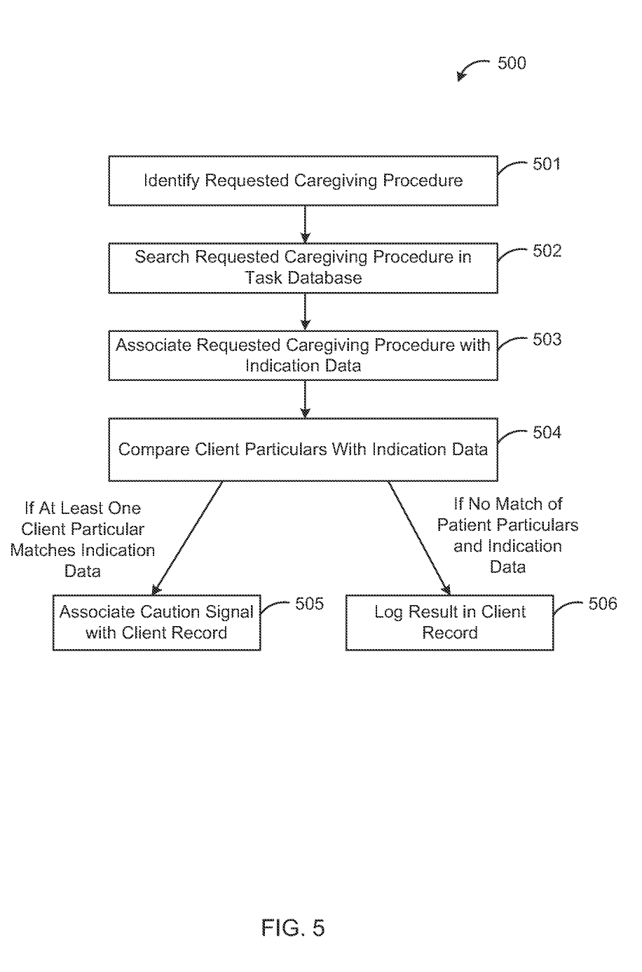

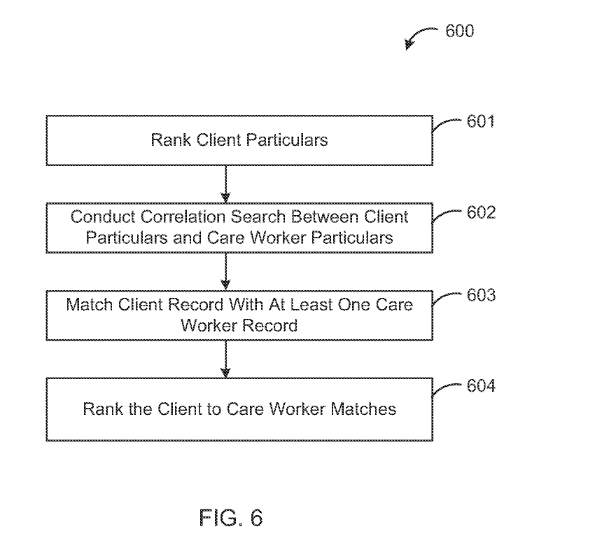

Client to care worker match module 303 may match a client record 205 to at least one care worker record 206 based on client particulars of a client record 205 and care worker particulars of a care worker record 206. Client to care worker match module 303 will be discussed in further detail having regard to FIG. 6. Task indicator module 304 within selection module 104 will be discussed in further detail having regard to FIG. 5.

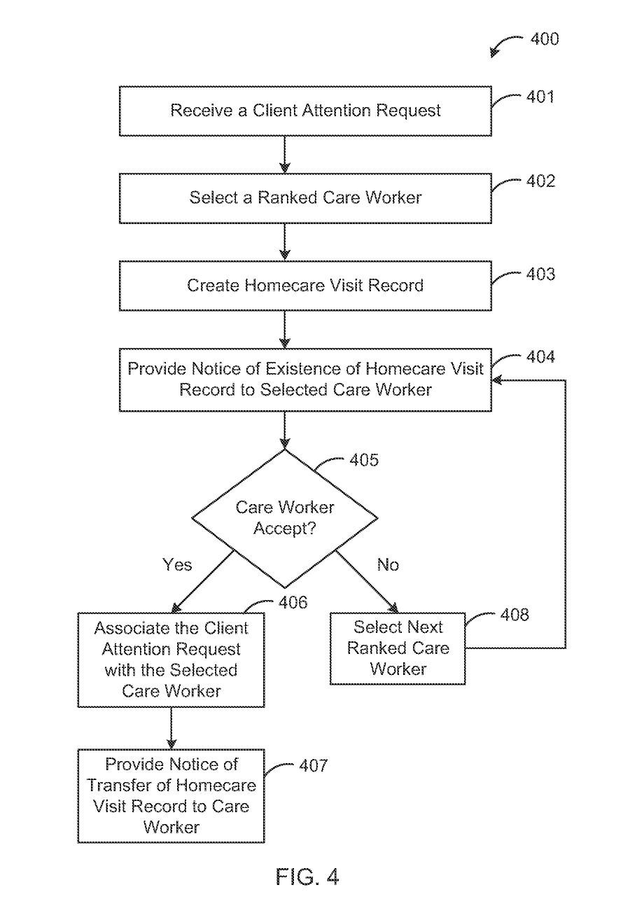

Reference is now made to FIG. 4, in which a method 400 for coordinating caregiving services for clients and care workers is illustrated. In an exemplary embodiment, processor 102 of caregiving services computing system 101 of FIG. 1 performs method 400 for coordinating caregiving services for clients and care workers.

At 401, processor 102 is configured to receive a client attention request 208. The client attention request 208 includes a requested caregiving procedure. The processor 102 associates client attention request 208 with a client record 205.

At 402, processor 102 selects a ranked care worker record 206 based on client attention request 208, client record 205, and care worker records 206. Ranking care worker records will be discussed in further detail having regard to FIG. 6.

At 403, processor 102 creates a homecare visit record 207 based on client attention request 208, client record 205, and selected care worker record 206 and stores the homecare visit record 207 in the database 103.

At 404, processor 102 provides notice of existence of homecare visit record 207 to the selected care worker associated with the care worker record 206.

At 405, when a care worker receives a notice of existence a homecare visit record 207, the care worker may review the contents of the homecare visit record 207, including information on the client and caution signals or alarm response signals, and decide whether the care worker is able to accept the homecare visit appointment. For example, in some cases, a care worker is unable to accept the homecare visit appointment because the caution signal indicates a caregiving procedure complexity that the care worker does not have the expertise or ability to handle.

If the selected care worker accepts the homecare visit appointment, at 406, processor 102 associates the client attention request 208 with the selected care worker record 206. At 407, processor 102 provides notice of transfer of the homecare visit record to the care worker.

If the selected care worker declines the homecare visit appointment, at 408, processor 102 selects the next ranked care worker and processor 102 provides notice of existence of a homecare visit record 207 to the next selected care worker.

Exemplary embodiment of method 400 for coordinating caregiving services for clients and care workers is akin to a “negotiation” involving the client, a selected care worker, and the system for coordinating caregiving services 100. A user 108 may submit a client attention request 208, on behalf of a client, to the system 100 for coordinating caregiving services. Similarly, user 108, acting on behalf of a client, may cancel the client attention request 208.

Similarly, a care worker may review the homecare visit record 207 and decide on the basis of the homecare visit record 207 contents whether the care worker is able to fulfil the client attention request 208. Allowing the client to request and cancel caregiving services and also allowing a ranked care worker the option to decline a homecare visit empowers both parties an ability to accept a caregiving service appointment within the system 100.

In another embodiment, the system 100 for coordinating caregiving services may provide the client and the selected care worker with access and continuous control of the homecare visit record. Access and continuous control of the homecare visit may be facilitated through user devices 107. Allowing access and continuous control of the homecare visit record facilitates interaction and empowerment of users 108.

Reference is now made to FIG. 5, in which a method 500 for identifying complexities and raising caution signals is illustrated. At 501, processor 102 identifies the requested caregiving procedure from client attention request 208.

At 502, processor 102 searches the requested caregiving procedure in task record 203. The task record 203 contains indication data, where the indication data includes information that may identify incompatibilities between the requested caregiving procedure and client particulars.

At 503, processor 102 associates the requested caregiving procedure with indication data of the task record 203. At 504, processor 102 compares client particulars of the client record 205 with indication data of task record 203.

At 505, if at least one client particular correlates to indication data, then processor 102 associates a caution signal with the client record 205. At 506, if the client particulars do not correlate to any of the indication data, then processor 102 logs the result in the client record 205.

For example, if a client attention request 208 indicates an appointment request for meal feeding assistance, processor 102 may search the meal feeding assistance request in task record 203 and associate the meal feeding assistance request with indication data. The indication data may provide guidelines for the meal feeding assistance and require that client particulars in client record 205 be considered. If, for example, a client particular in client record 205 indicates that a client is diabetic, the method 500 may raise a flag cautioning a care worker of a potential dietary restriction for the client of client record 205.

Reference is now made to FIG. 6, in which a method 600 for ranking client to care worker matches is illustrated. At 601, processor 102 ranks client particulars of the client record 205. In an embodiment, processor 102 may rank client particulars based on user 108 input. For example, a client may have a strong preference for interacting with a care worker who speaks French and who is female. Processor 102 may rank client particular data fields relating to language preference and care worker gender preference higher than other client particular data fields. In another example, a client attention request 208 entered for a client recovering from surgery may require assistance with daily exercises. Processor 102 may rank client particular data fields relating to care worker expertise preference higher than other client particular data fields.

At 602, processor 102 conducts a correlation search between client particulars of the client record 205 and care worker particulars of the care worker record 206. In an embodiment, the correlation search may identify client particulars of client record 205 that match or are related to care worker particulars of care worker record 206.

At 603, processor 102 matches client record 205 with at least one care worker record 206. In an embodiment, matching client record 205 to a care worker record 206 occurs when at least one client particular of the client record 205 is equal to at least one care worker particular of a care worker record 206. In another embodiment, matching a client record 205 to a care worker record 206 may occur when a client particular of client record 205 is correlated to a care worker particular of care worker record 206. For example, a correlation search may identify a client particular indicating that a client has hearing loss and match the client of client record 205 with a care worker of care worker record 206 containing a care worker particular indicating proficiency in sign language.

At 604, based upon the ranking of the client particulars at 601, processor 102 ranks client record 205 to care worker 206 matches. For example, a client record 205 to care worker 206 match based upon a care worker's French speaking skills and gender may rank higher than matches based upon other client particular to care worker particular matches.

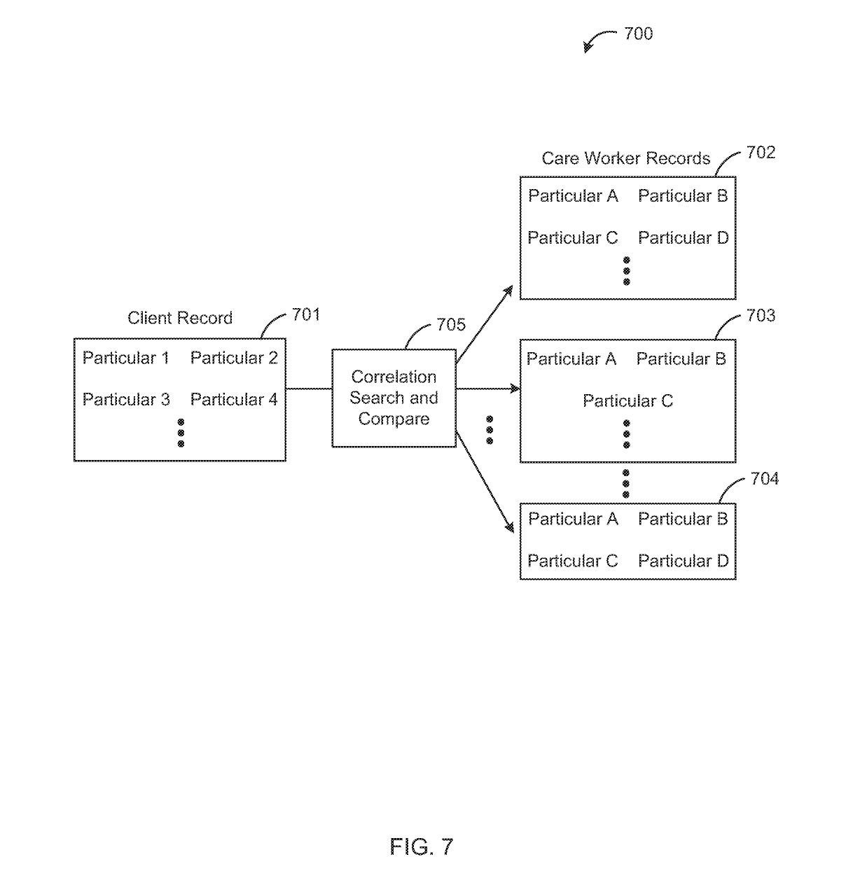

Reference is now made to FIG. 7, in which a method for ranking client to care worker matches 600 is illustrated in an exemplary embodiment. Client record 701 includes multiple particular data fields. Numerous care worker records 702, 703, 704, includes multiple particular data fields. Processor 102 conducts a correlation search and compare 705 between client record 701 and care worker records 702, 703, 704.

In an embodiment, processor 102 may conduct a correlation search and compare of client particulars of client record 701 with any and all care worker particulars of care worker records 702, 703, 704. In another embodiment, processor 102 may only conduct a correlation search and compare of a particular of client record 701 with a particular data field in the care worker records 702, 703, 704.

Reference is now made to FIG. 8, in which a method for tracking time duration of caregiving procedures 800 is illustrated. At 801, processor 102 identifies the requested caregiving procedure from the client attention request 208.

At 802, processor 102 creates time parameters for requested caregiving procedure. In an embodiment, the time parameters may be stored in homecare visit record 207. In an embodiment, user 108 may manually enter the time parameters for the requested caregiving procedure. In a further embodiment, time parameters are created based on analysis of aggregate time parameter data obtained from all home care visit records 207 for a same or similar requested caregiving procedure.

At 803, processor 102 begins duration timer, which tracks the elapsed time of the caregiving procedure. In an exemplary embodiment, care worker user 108c accesses the client record 205 or homecare visit record 207 using user device 107 when a scheduled caregiving procedure begins. Care worker user 108c closes the client record 205 or homecare visit record 207 using user device 107 when the caregiving procedure is complete. The duration timer starts when client record 205 or homecare visit record 207 is accessed. The duration timer stops when client record 205 or homecare visit record 207 is closed. In another exemplary embodiment, the duration timer may be started and stopped by selecting a button on user device 107.

At 804, processor 102 compares the elapsed time to time parameters. If caregiving procedure is not yet complete and the elapsed time does not exceed the time parameters, duration timer continues to keep track of elapsed time of the caregiving procedure.

At 805, if caregiving procedure is not yet complete and the elapsed time exceeds time parameters, processor 102 creates a communication channel between care worker of care worker record 206 and administrator user 108a. A communication channel may be a telephone call, cellular text message, or any other mode of communication between two parties.

At 806, if a communication channel between care worker of care worker record 206 and administrator user 108a cannot be established, processor 102 transmits an alarm response signal at 806 and stores alarm response signal in care worker record 206 and homecare visit record 207. In an embodiment, an alarm response signal in a care worker record 206 may indicate to correlation search at 602 that the care worker of care worker record 206 may not be available for other caregiving appointments.

In an embodiment, when processor 102 transmits an alarm response signal at 806, administrator user 108a will attempt to call care worker of care worker record 206 and to establish a communication channel. If a communication channel cannot be established, administrator user 108a will attempt to call client of client record 205. If a communication channel cannot be established, administrator user 108a may dispatch emergency personnel.

At 808, if a communication channel is established with either the care worker of care worker record 206 or client of client record 205, administrator user 108a may manually update or modify the time parameters. At 810, administrator user 108a may update care worker record 206 and homecare visit record 207. Processor 102 stores alarm response signal in care worker record 206 and homecare visit record 207.

At 809, if the caregiving procedure is complete, processor 102 stores elapsed time in homecare visit record 207.

In an embodiment, user device 107 may include a panic button option on each screen. Once clicked by the care worker of care worker record 206, processor 102 creates a communication channel between the care worker of care worker record 206 and administrator user 108a. In another embodiment, once a care worker or care worker record 206 clicks a panic button on user device 107, a “high priority email” may be sent to administrator user 108a to notify that there may be an issue with the client, either medical or abusive.

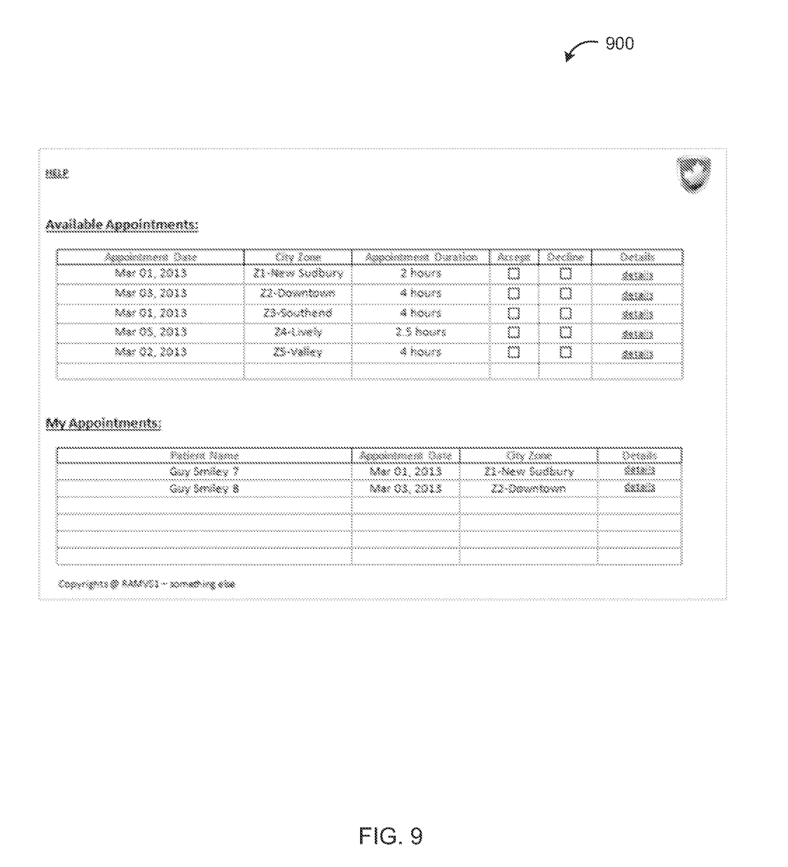

Reference is now made to FIG. 9, which is a screenshot 900 of an example user interface where a care worker may review, at 405, contents of a homecare visit record and decide whether the care worker is able to accept a homecare visit appointment. The user interface may allow a care worker of care worker record 206 to review the contents of home care visit record 207, including information of the client record 205 and caution or alarm response signals.

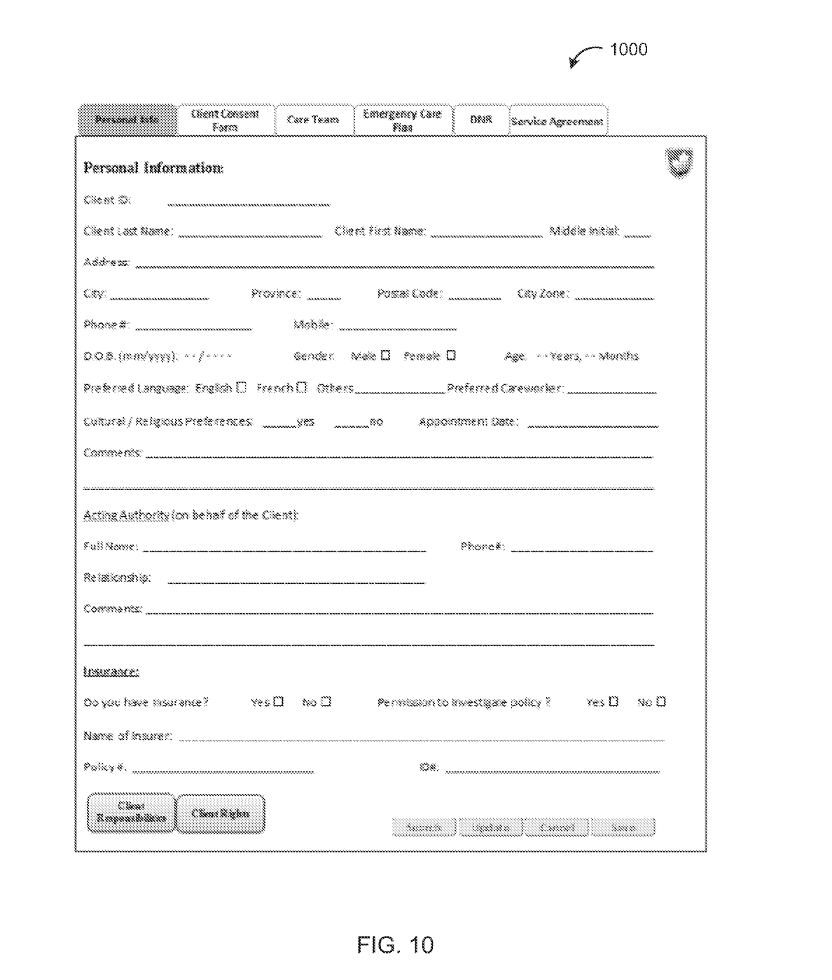

Reference is now made to FIG. 10, which is a screenshot 1000 of an example user interface for accessing client data of client record 205 by user 108 on user device 107. The data fields of screenshot 1000 are examples of client particulars of client record 205.

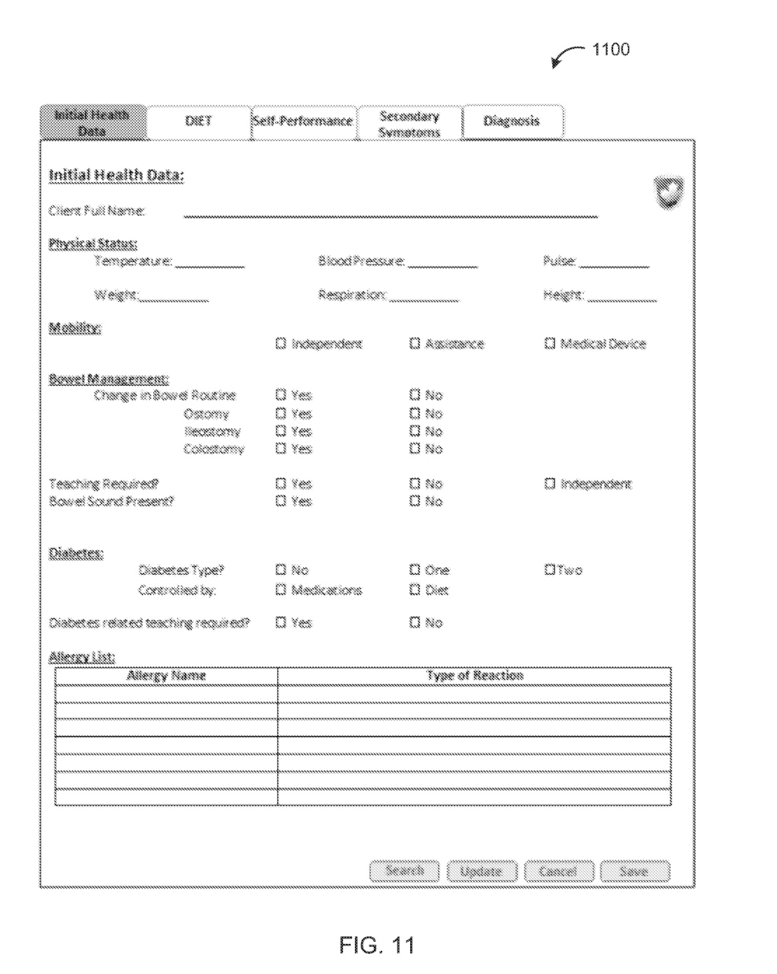

Reference is now made to FIG. 11, which is a screenshot 1100 of an example user interface for accessing client data of client record 205 by user 108 on user device 107. The data fields of screenshot 1100 are examples of client particulars of client record 205 representing client health related data.

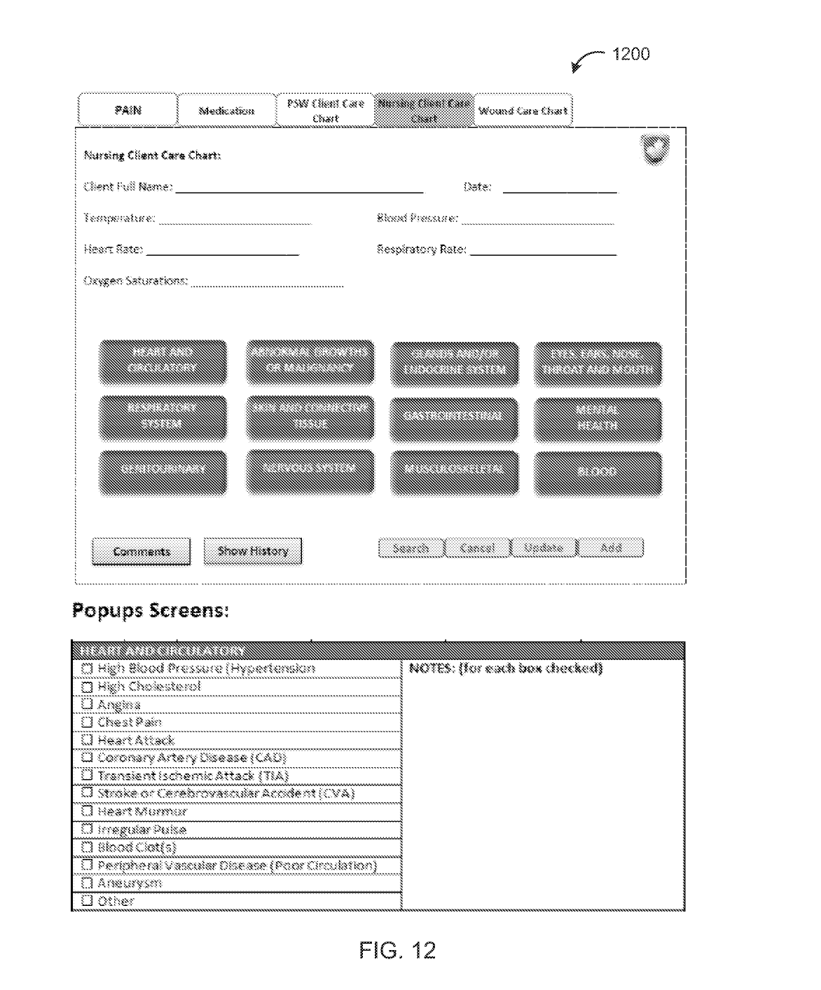

Reference is now made to FIG. 12, which is a screenshot 1200 of an example user interface for accessing client data of client record 205 by user 108 on user device 107. The data fields of screenshot 1200 are examples of client particulars of client record 205 representing client care charts. In an exemplary embodiment, the client particulars illustrated in screenshot 1200 may for example, at 504, result in processor 102 associating a caution signal with client record 205.

The present invention has been described here by way of example only. Various modification and variations may be made to these exemplary embodiments without departing from the spirit and scope of the invention, which is limited only by the appended claims. Also, in the various user interfaces illustrated in the figures, it will be understood that the illustrated user interface text and controls are provided as examples only and are not meant to be limiting. Other suitable user interface elements may be possible.

1. A method for coordinating caregiving services for clients and care workers using a caregiving services computing system comprising a database and processor, the method comprising:
storing at least one client record containing client particulars and at least one care worker record containing care worker particulars in the database;
upon receiving a client attention request:
associating the client attention request with a client record, wherein the client attention request includes a requested caregiving procedure,
selecting a ranked care worker record based on the client attention request, the client record, and the care worker records,
creating a homecare visit record based on the client attention request, the client record, and the selected care worker record and storing the homecare visit record in the database;
wherein the homecare visit record further comprises a record of caution signals and alarm response signals,
providing notice of existence of the homecare visit record to the selected care worker associated with the care worker record,
upon receiving a care worker accept decision signal:
associating the client attention request with the selected care worker record,
providing notice of transfer of the homecare visit record to the care worker associated with the selected care worker record.

2. The method of claim 1, wherein the method further comprises:
selecting a next ranked care worker record only where:
the selected care worker transmits a care worker decline decision signal.

3. The method of claim 1, wherein the method further comprises:
providing the client and the selected care worker with access and continuous control of the homecare visit record.

4. The method of claim 1, the method further comprises,
creating time-parameters for the requested caregiving procedure;
initiating a duration timer when the requested caregiving procedure begins;
comparing the elapsed time to the time-parameters;
transmitting an alarm response signal to an administrator and storing the alarm response signal in the homecare visit record only where:
the service is not complete and the elapsed time is greater than the time-parameters for the requested health care procedure;

5. The method of claim 4, wherein creating time-parameters comprises:
manually specifying the new time-parameters.

6. The method of claim 4, wherein creating time-parameters comprises:
identifying amongst a plurality of homecare visit records all time-parameters associated with the requested caregiving procedure;
computing a statistical mean of all time-parameters associated with the requested caregiving procedure.

7. The method of claim 4, wherein transmitting an alarm response signal to an administrator further comprises:
creating a communication channel between the care worker and the administrator.

8. The method of claim 7, wherein the method further comprises facilitating a set of panic and communication buttons.

9. The method of claim 1, wherein selecting a ranked care worker record comprises:
searching the requested caregiving procedure in a task record,
wherein the task record associates the requested caregiving procedure with indication data;
comparing the client particulars to the indication data of the task record;
associating a caution signal with the client record only where:
at least one client particular correlates to at least one particular of the indication data of the task record.

10. The method of claim 9, wherein the method further comprises,
ranking the client particulars;
conducting a correlation search between the client particulars of the client record and the care worker particulars of the care worker records;
matching a client record to at least one care worker record;
based upon the ranking of the plurality of client particulars:
ranking the care worker records.

11. The method of claim 10, wherein matching a client record to at least one care worker record occurs when:
at least one client particular is equal to at least one care worker particular in a care worker record.

12. The method of claim 9, wherein the indication data comprises:
information identifying incompatibilities between the requested caregiving procedure and client particulars.

13. A non-transitory computer readable medium for performing the steps of the method of claim 1.

14. A system for coordinating caregiving services for clients and care workers, the system comprising:
a database for storing at least one client record containing client particulars and at least one care worker record containing care worker particulars;
a processor and selection module coupled to the database, said processor operable to:
upon receiving a client attention request:
associate the client attention request with a client record, wherein the client attention request includes a requested caregiving procedure,
select a ranked care worker record based on the client attention request, the client record, and the care worker records,
create a homecare visit record based on the client attention request, the client record, and the selected care worker record and store the homecare visit record in the database;
wherein the homecare visit record further comprises a record of caution signals and alarm response signals,
provide notice of existence of the homecare visit record to the selected care worker associated with the care worker record;
upon receiving a care worker accept decision signal:
associate the client attention request with the selected care worker record,
provide notice of transfer of the homecare visit record to the care worker associated with the selected care worker record.

15. The system of claim 14, wherein the processor is further operable to:
select a next ranked care worker record only where:
the selected care worker transmits a care worker decline decision signal.

16. The system of claim 14, wherein the processor is further operable to:
provide the client and the selected care worker with access and continuous control of the homecare visit record.

17. The system of claim 14, wherein the processor is further operable to:
create time-parameters for the requested caregiving procedure;
initiate a duration timer when the requested caregiving procedure begins;
compare the elapsed time to the time-parameters;
transmit an alarm response signal to an administrator and store the alarm response signal in the homecare visit record only where:
the service is not complete and the elapsed time is greater than the time-parameters for the requested health care procedure;

18. The system of claim 17 wherein creating time-parameters comprises:
manually specifying the new time-parameters.

19. The system of claim 17, wherein creating time-parameters comprises:
identifying amongst a plurality of homecare visit records all time-parameters associated with the requested caregiving procedure;
computing a statistical mean of all time-parameters associated with the requested caregiving procedure.

20. The system of claim 17, wherein transmitting an alarm response signal to an administrator further comprises:
creating a communication channel between the care worker and the administrator.

21. The system of claim 20, wherein the processor is further operable to:
facilitate a set of panic and communication buttons.

22. The system of claim 14, wherein selecting a ranked care worker record comprises:
searching the requested caregiving procedure in a task record,
wherein the task record associates the requested caregiving procedure with indication data;
comparing the client particulars to the indication data of the task record;
associating a caution signal with the client record only where:
at least one client particular correlates to at least one particular of the indication data of the task record.

23. The system of claim 22, wherein the processor is further operable to:
rank the client particulars;
conduct a correlation search between the client particulars of the client record and the care worker particulars of the care worker records;
match a client record to at least one care worker record;
based upon the ranking of the plurality of client particulars:
rank the care worker records.

24. The system of claim 23, wherein matching a client record to at least one care worker record occurs when:
at least one client particular is equal to at least one care worker particular in a care worker record.

25. The method of claim 22, wherein the indication data comprises:
information identifying incompatibilities between the requested caregiving procedure and client particulars.
